(19) United States
(12) Patent Application Publication
(10) Pub. No.: US 2003/0161883 A1
(43) Pub. Date: Aug. 28, 2003

(54) Composition consisting of an active ingredient and a therapeutically active delivery system, especially in the treatment of angiogenesis

(76) Inventors: Hilmar Meek Warenius, Wirral (GB); Robert Alexander Walker Johnstone, Bebington (GB)

Correspondence Address: ROTHWELL, FIGG, ERNST & MANBECK, P.C., 1425 K STREET, N.W., SUITE 800, WASHINGTON, DC, 20005, US

(21) Appl. No.: 10/181,494

(22) PCT Filed: Jan. 22, 2001
(86) PCT No.: PCT/GB01/00238

(30) Foreign Application Priority Data
Jan. 21, 2000 (GB) 0001481.1

(51) Int. Cl. A61K 31/704; A61K 31/4745; A61K 31/337; A61K 9/14

(52) U.S. Cl. 424/486; 514/34; 514/283; 514/449

(57) ABSTRACT

Provided is a composition comprising: (a) an active component having a therapeutic and/or diagnostic activity; and (b) a delivery component for facilitating delivery of the active component, wherein the delivery component has a therapeutic and/or diagnostic activity.

The present invention concerns a system for the delivery of a medicament, pharmaceutical or drug. In particular, the invention concerns a delivery system comprising a delivery agent and a medicament, wherein the delivery agent itself has medicinal activity. The invention also relates to methods of forming the delivery system and uses for the delivery system.

Cancers are known to arise as a result of a local clonal proliferation of malignantly transformed cells. The size to which a local tumour can grow is limited by the availability of nutrients and oxygen which must reach all the cells in order for them to remain healthy and grow effectively. In tissues, the oxygen diffusion limit is 100-200 μm, which is equivalent to a depth of between 3 and 5 concentric cellular lines around a single blood vessel. Therefore, in order for local tumour proliferation to proceed beyond a certain size, it is necessary for the tumour to develop a blood supply (Folkman, 1989). This neovascularisation is termed angiogenesis. The first consequence of angiogenesis is that it enables tumours to grow progressively to a potentially large size without restraint in the local site in which they arise. A further consequence is that the ingrowing blood vessels provide channels for the dissemination of cells from the primary tumour throughout the body. The cells, which spread from the primary site can lodge in other distant sites and themselves proliferate to form new (metastatic) tumours. In order to grow beyond a very limited size these metastatic tumours must themselves stimulate angiogenesis. Methods of inhibiting angiogenesis have thus been actively sought over the past three decades as a means of both diminishing the volume of primary tumours, preventing the spread of metastatic tumour cells and inhibiting the growth of metastases (for review see Gasparini, 1999).

Several compounds of different classes have been investigated as putative anti-angiogenic agents, with some of them now undergoing clinical trials. Many anti-angiogenic therapy agents have been developed (for review see Gasparini 1997) and some have progressed to the stage of clinical trials. These can be broadly classed as biological agents and chemical agents. The biological agents include angiostatin, endostatin, thrombospondin-1, platelet factor 4, interferons, interleukin-12, antibodies to angiogenic peptides and integrins, anti-angiogenic vaccines, novel anti-angiogenic peptides and gene therapy. The chemical agents include suramin and many of its variants plus a variety of other chemicals, including some polysulphonic acids. Certain anti-angiogenic agents, such as polysulphonates have also been found to possess anti-HIV activity. A list of the above agents is given below with references to their testing and use.

1. Sulphonic acid polymers, including poly[styrene sulphonic acid] (PSS); poly[anethole sulphonic acid] (PAS); poly[vinyl sulphonic acid] (PVS); and poly[2-acrylamido-3-methyl-1-propane sulphonic acid] (PAMPS), against angiogenesis (see S. Lienks, J. Neyts, B. Degreve and E. De Clerq, Oncol. Res., 1997, 9, 173-181).

2. The same agents as for 1 have also been tested against HIV (see G. Tan, A. Wickramasinghe, S. Verma, S. Huges, J. Pezzuto, M. Baba and P. Mohan, Biochim. Biophys. Acta, 1993, 1181, 183-188).

3. The same agents as for 1, plus poly[vinylphosphate] (PVP) and poly[4-hydroxyphenylstyrene] (PHP) have been tested against HIV (see S. Ikeda, J. Neyts, S. Verma, A. Wickramasinghe, P. Mohan and E. De Clerq, Antimicrob. Agents Chemother., 1994, 38, 256-259).

4. Heterocyclic analogues of suramin against angiogenesis (see F. Manetti, V. Capello, M. Botta, F. Correli, N. Mongelli, G. Biasoli, A. Borgia and M. Ciomei, Bioorg. Med. Chem., 1998, 6, 947-958).

5. Suramanin analogues against angiogenesis (see A. Gagliardi, M. Kassack, A. Kreimeyer, G. Muller, P. Nickel and D. Collins, Cancer Chemother. Pharma, 1998, 41, 117-124).

6. Suramin and more analogues against angiogenesis (see R. Lozano, M. Jimenez, J. Santoro, M. Rico and G. Gallego, J. Mol. Biol., 1998, 281, 899-915).

7. Suramin and suaradistas against angiogenesis (see A. Gagliardi, H. Hadd and D. Collins, Cancer Res., 1992, 52, 5073-5075).

8. Suramin analogues against angiogenesis (see A. Firschung, P. Nickel, P. Mora and B. Allolio, Cancer Res., 1995, 55, 4957-4961).

9. Flavone acetic acid (FAA) against angiogenesis (see L-M. Chang, Z-F. Xu, B. Gummer, B. Palmer, W. Joseph and B. Baugles, Brit. J. Cancer, 1995, 72, 339-343).

10. Fumagillin and AGM 1470, metabolites from Aspergillus fungi against angiogenesis (see D. Infber, T. Fujita, S. Kishimoto, K. Sudo, T. Kanamaru, H. Brem and J. Folkman, Nature, 1990, 348, 555-55).

11. 2-methoxy-estradiol and taxol against angiogenesis (see N. Klauber, S. Parangi, E. Flynn, E. Hamel and R. d'Amoto, Cancer Res., 1997, 57, 81-86).

12. Retinoic acid against angiogenesis (see M. Lingen, P. Polverini and N. Buck, Lab. Invest., 1996, 74, 476-483); and

13. Polymers (PSS), (PAMPS), (PVS) and (PAS) against HIV (see P. Mohan, D. Schols, M. Baba and E. De Clerq, Antiviral Res., 1992, 18, 139-150).

The suramins (suramin itself and its analogues) disclosed in the above references are discrete single molecules, based on the following structure:

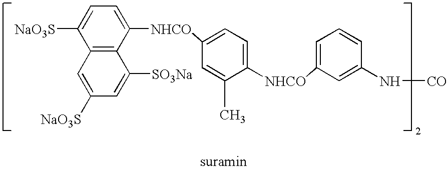

The compounds PSS, PVS, PAS, PAMPS, PHP and PVP are all simple homopolymers having no drug carrying capacity, and have not been covalently linked to drugs, nor been used to provide imaging facilities, nor been used with fluorescent molecules.

The other types of anti-angiogenesis agents disclosed in the above references are all discrete single molecules.

A limitation of anti-angiogenesis therapy is that, whilst the available agents have diverse efficacy during the process of carcinogenesis, none of the available agents can completely block the angiogenic switch in pre-malignant conditions, block the growth of small tumours or induce complete remission of advanced tumours (Bergers et al, 1999). This finding fits well with an understanding of the action of anti-angiogenic agents in that these agents will lead to the death of tumour cells dependent upon a vascular supply but there will still be some remaining viable tumour cells that can regrow. Thus anti-angiogenesis treatment is likely to need to be maintained for long periods of time if not for a lifetime. For this reason, to obtain maximum effect, anti-angiogenesis drugs would need to be prescribed with cytotoxic drugs and/or radiotherapy.

A further consequence of the neovascularisation of tumours is that, despite establishing a blood supply, the tumour cells themselves cannot be adequately accessed by anticancer agents. Thus a major reason why cytotoxic drugs are frequently ineffective in the majority of human solid tumours is that the full cytotoxic action is not delivered directly to the tumour cells. The inability to fully access tumour cells with therapeutic agents is not limited to present therapy and is equally as likely to be a problem with future therapies even though such treatments may be much more accurately targeted to the specific tumour (Jain 1998). The inability of present or future anticancer agents to successfully access all the malignant cells in a tumour is of particular importance in metastatic cancer, which is responsible for three out of four cancer deaths and for which the only possible treatment is systemic therapy with cytotoxic drugs or the more recent biological agents (Beardsley 1994). Jain (1998) has provided a number of reasons which explain why the delivery of therapeutic agents to the cells within a tumour is so poor:

(i) although tumour blood vessels arise from well-organised host vessels, the new blood vessels in tumours are disorganised in their growth, structure and function resulting in temporal and spatial heterogeneity of blood flow;

(ii) there is tremendous heterogeneity of the permeability of the vessel wall of blood vessels in tumours with the result that, in some regions of the tumour, therapeutic agents cannot escape from the blood vessel to gain access to the adjacent tumour cells;

(iii) solid tumours exert interstitial hypertension and thus convection (pressure-driven bulk flow across blood vessel walls) is reduced.

Methods and agents for packaging and delivering therapeutic and diagnostic agents are known. Previously these methods have generally been developed to facilitate the uptake and delivery of agents which are substantially water-insoluble. Methods have included encapsulation in micelles and lipid-like materials. These systems have the disadvantage that they are dynamic and tend to release the encapsulated agent prematurely. An improved delivery system is disclosed in published international patent application PCT/GB98/03046. This document discloses an amphiphilic polymer comprising hydrophylic and hydrophobic units. The polymer is indicated to be useful for packaging therapeutic and diagnostic agents, such as delivering therapeutic inhalants to the lungs, and Taxol to cancer cells. However, improved packaging and delivery systems are required, in particular for combination therapy requiring two or more active agents, such as an anti-angiogenesis agent in combination with a cytotoxic agent.

Thus, in order to more effectively deliver anticancer agents to the individual cancer cells that comprise a solid tumour, a method of carrying the anti-cancer agent using an agent capable of selectively destroying tumour blood vessels and thus allowing free access of the anticancer agent to the cancer cells, is required.

It is an aim of the present invention to overcome the disadvantages associated with the above known products and methods. It is a further aim of the present invention to provide an improved packaging and/or delivery system for combination therapy With this in mind a more specific aim of the present invention is to provide an effective way to combine an anti-angiogenic agent with a cytotoxic drug, since there is at present no effective method of accomplishing this. A still further aim of the present invention is to provide an improved method of delivering a cytotoxic drug to eradicate the remaining cancer cells that have survived anti-angiogenic therapy.

Accordingly, the present invention provides a composition, which composition comprises:

(a) an active component having a therapeutic and/or diagnostic activity; and

(b) a delivery component for facilitating delivery of the active component,

wherein the delivery component has a therapeutic and/or diagnostic activity.

The present composition can be used in methods of treating mammals, especially humans, for diseases or conditions associated or arising out of angiogenesis.

The activity of the components in the composition is not particularly limited and both components may have the same activity or different activities. The composition as presently claimed is preferably used in combination therapy such as therapy combining anti-angiogenic activity with cytotoxic activity. It is especially preferred that the delivery component has anti-angiogenic activity and the active component has cytotoxic activity.

The delivery component may serve to facilitate delivery of the active component by any means. However it is particularly preferred that the delivery component solubilises the active component, or provides a hydrophobic region, cavity or pocket to shield the insoluble active component from the hydrophilic surroundings.

An important advantage of the present invention is that it provides a combination of two or more therapeutic agents in a simpler package than was previously possible, since the packaging polymer itself acts as a therapeutic agent. Previously, no significant practical consideration had been given to providing a packaging component with additional therapeutic activity, possibly due to practical problems of maximising therapeutic effect, whilst maintaining effective packaging properties.

The present invention also offers several important specific advantages:

1. In one preferred aspect it can provide a solution to the problem of dealing with tumour cells not killed by an anti-angiogenic agent, by providing an anti-angiogenic agent that is also a drug solubilising or drug-carrying polymer. Thus both anti-angiogenic activity and cytotoxic activity can be delivered simultaneously to the tumour. Examples of cytotoxic drugs that could be carried are taxanes such as Taxol® (paclitaxel) or Taxotere® (docetaxel);

2. In a further preferred aspect it can provide a solution to the difficulty in delivering anticancer therapy to tumour cells because of difficulties imposed by the tumour vasculature, by delivering the anticancer therapy in a vehicle, which is itself anti-angiogenic and will thus disrupt the tumour blood vessels or prevent new blood vessel formation during tumour re-growth following cell killing by the delivered cytotoxic drug. This will carry the anticancer therapy more directly to the target cells than is presently possible.

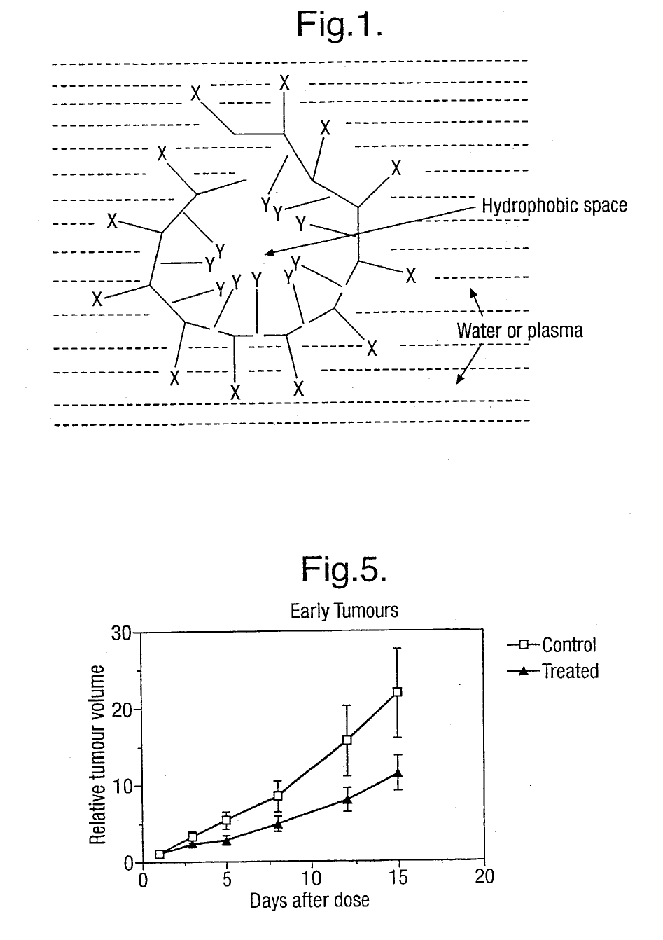

The delivery component is preferably a polymer comprising hydrophilic and hydrophobic groups or units. The polymer is thus generally a co-polymer formed from a monomer comprising a hydrophilic group and a co-monomer comprising a hydrophobic group. However, the polymer may alternatively be formed from a single monomer which comprises both a hydrophilic and a hydrophobic group. The groups may form part of the polymer backbone, or may be pendant from the backbone. The polymer may be a random co-polymer, a graft co-polymer or a block co-polymer. The polymer structure is preferably arranged such that a hydrophobic region, domain, channel, cavity or pocket is present or can be formed for trapping or encapsulating an active component (see FIG. 1).

In a preferred embodiment, the purpose of the polymer structure is to provide a water-soluble polymer having inherent anti-angiogenic properties, which is also able to deliver normally water-insoluble chemotherapeutic agents to its site of action. A further purpose of the polymer is to provide a water-soluble, anti-angiogenic agent capable of carrying covalently-linked water-soluble prodrugs to tumours where enzymatic cleavage of the covalent link releases an activated water-soluble drug in close proximity to the tumour cells.

In a further preferred embodiment, the purpose of the polymer structure is to provide an anti-HIV agent that can, in addition, carry anti-HIV drugs (such as relatively non-water soluble variants of AZT and other anti-HIV agents) to the HIV virus providing a doubly damaging therapeutic effect.

The constituents of the polymer provided in this invention are preferably selected from water soluble and water insoluble agents where at least one agent has known activity, such as anti-angiogenic or anti-HIV activity. In the case of anti-angiogenesis, naphthalene and styrene polysulphonates are preferably combined because each independently has been previously shown to have anti-angiogenic activity and the combination of the two (a water insoluble and a water soluble moiety) in a copolymer enables the production of an active copolymer that is also capable of acting as a drug carrier. In order to provide a non-water solubilising component in the copolymer, sulphonate groups are preferably omitted from the naphthyl moiety.

In the case of anti-HIV activity, it is preferred that polystyrene sulphonate is combined with water insoluble moieties (for example naphthyl) in order to provide an anti-HIV agent that is capable of simultaneously delivering a second independently-acting anti-HIV agent.

The invention also provides a means of treating benign conditions in which neovascularisation is a problem. These include: vascular stents in arterioschlerosis; diabetic retinopathy; conditions following surgery for glaucoma or following other ophthalmic surgery including macular degeneration; vascular causes of blindness including macular degeneration; dermatological conditions such as psoriasis and cosmetic conditions affecting ageing of the skin such as telangiectasis. As with other applications the co-polymer can be used alone as an anti-angiogenic agent in these cases or combined with another therapeutic agent which is hydrophobic in nature.

An example of a typical preferred polymer is provided in FIG. 1. Here water-solubilising groups (X) and hydrophilic groups (Y) can adopt a structure in water having a hydrophobic centre associated with a hydrophilic region (for example, surrounded by a hydrophilic shell).

The hydrophobic sections are capable of holding several drug molecules (preferably from 6-20) in a co-polymer of molecular weight, Mᵣ in the range of generally about 10,000-220,000, more usually 10,000-120,000. The drug molecules may be held only by intermolecular non-bonding forces or may be covalently attached to the polymer. One typical copolymer has X=SO₃Na, or phenyl-SO₃Na and Y=2-naphthyl. This type of co-polymer may be employed with a variety of water-insoluble drugs (e.g. porphyrins, taxol, or busulfan).

Independently of its dual acting capacity as a carrier for anti-cancer and/or anti-HIV agents the copolymer on its own is active against angiogenesis and/or HIV. By themselves, these copolymers are active therapeutically and/or diagnostically, being capable, for example, of inhibiting angiogenesis and HIV. Although some polymers (but not copolymers) with similar anti-angiogenesis properties have been described in the prior art, these agents are not capable of carrying chemotherapeutic drugs into aqueous solution or plasma By utilising the two properties together within the same molecule (drug-carrying capacity and activity, such as anti-angiogenesis or anti-HIV activity) there is created an improved utility for the copolymers, viz., inhibiting angiogenesis or HIV while at the same time delivering therapeutic doses of drugs, such as cytotoxic drugs to effect the killing of cells, where anti-angiogenesis is occurring or in patients suffering from HIV.

The polymers preferably comprise a backbone, from which are appended groups with desirable properties. The backbone may comprise, for example, a polymethylene backbone. The polymers may be of random, graft or block type. Thus, when the polymers comprise groups appended from the backbone, they may comprise any one or more of the following units in any proportion or configuration:

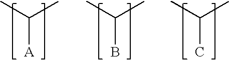

wherein A is preferably a hydrophilic group, such as the following:

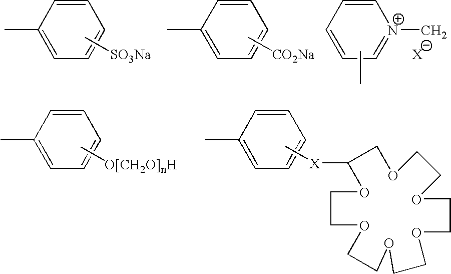

B is preferably a hydrophobic group such as the following:

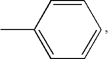

and other aromatics, heteroaromatics, alkyls etc. and C is preferably a hydrophilic or hydrophobic group and is used to supply other desirable properties. For example, the incorporation of the following groups allows their use in magnetic imaging instruments (to use F or similar atoms suitable for nuclear magnetic resonance spectroscopy):

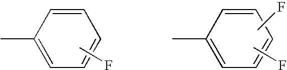

Alternatively C may comprise the following and similar fluorescent groups to monitor the presence and amount of polymer:

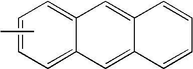

Preferably the polymer comprises both A and B groups, but it may comprise only A groups, only B groups or only C groups, if desired. More preferably, the polymer comprises all of groups A, B and C. However, the polymer may comprise groups A and C (no B) or groups B and C (no A) if desired. It will be appreciated that if the polymer comprises no A groups, then the hydrophilic groups are present within the backbone of the polymer. Similarly, if the polymer comprises no B groups, then the hydrophobic groups are present within the backbone of the polymer. If the polymer comprises only C groups then both the hydrophilic and hydrophobic groups are present in the backbone.

Thus, in one preferred embodiment of the present invention, the polymer may comprise a compound having a structure as exemplified below:

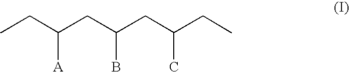

These particular polymers do not need to consist of the above structure (I) exactly, since this is only a representation. They need only comprise units from which groups A, B and C are pendant. Thus, the A, B and C groups may be arranged randomly throughout the polymer, or in blocks, or in some pre-arranged pattern, such as alternating groups, as in the above formula (I). This also applies to the polymers of the invention described above which only comprise A, B, C, A and B, B and C, or A and C groups.

Preferably, the polymers are prepared by reacting mixtures of suitable styrenes. Preferred ratios for A to B are from 1:1 to 1:20 or 20:1. Preferred ratios for (A+B) to C are from 1:1 to 50:1.

In addition to operating as a dual therapeutic agent capable of itself damaging the therapeutic target and also delivering a second therapeutic agent (which is preferably non-water soluble), the delivery component such as the polymers (preferably co-polymers) described here can be used to carry any drug by attaching it via a hydrolisable linkage to one of the moieties in the copolymer. This approach can also be used for drug latentiation. In the technique of drug latentiation a therapeutic agent is carried to the target site in an inactive form and then activated under the particular conditions pertaining at the site of the therapeutic target. The limitations of this approach in the past have been the absence of specific activating enzymes in high concentration at the target site. For this reason techniques of targeting activating enzymes using antibodies attached to enzynes (ADEPT) have been attempted.

More recently, attempts have been made to deliver the genes which produce activating enzymes by similar approaches. In this invention it is not necessary to deliver activating enzymes to the site where prodrug activation is required. Instead, use may be made of the destructive effect of the anti-angiogenic effect of the present copolymer which by destroying cells releases large amounts of usually unavailable intracellular enzymes present in the endothelial cells and adjacent stromal and tumour cells forming the new blood vessels at the desired target site within the tumour. For example enzymes such as esterases contained in lysosomes are only released when a cell is damaged. Linkages such as esters may therefore be used to attach drugs such as alkylating agents in latent form. Destruction of neovascularising endothelial cells within the target tumour may disrupt cellular lysosomes releasing esterases which break the hydrolysable ester linkage and release the active agent.

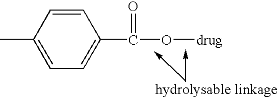

The invention will now be described in further detail by way of example only, with reference to the following specific embodiments.

### EXAMPLES

### Example 1

### Effect of Polystyrene Sodium sulphonate-co-vinyl naphthalene (TR01, TheraSol) on an in-vitro Model of Angiogenesis

This experiment describes the inhibitory effect of TR01 on new blood vessel formation (angiogenesis) in an in-vitro co-culture of human umbilical vein endothelial cells (HUVECs) and human fibroblasts.

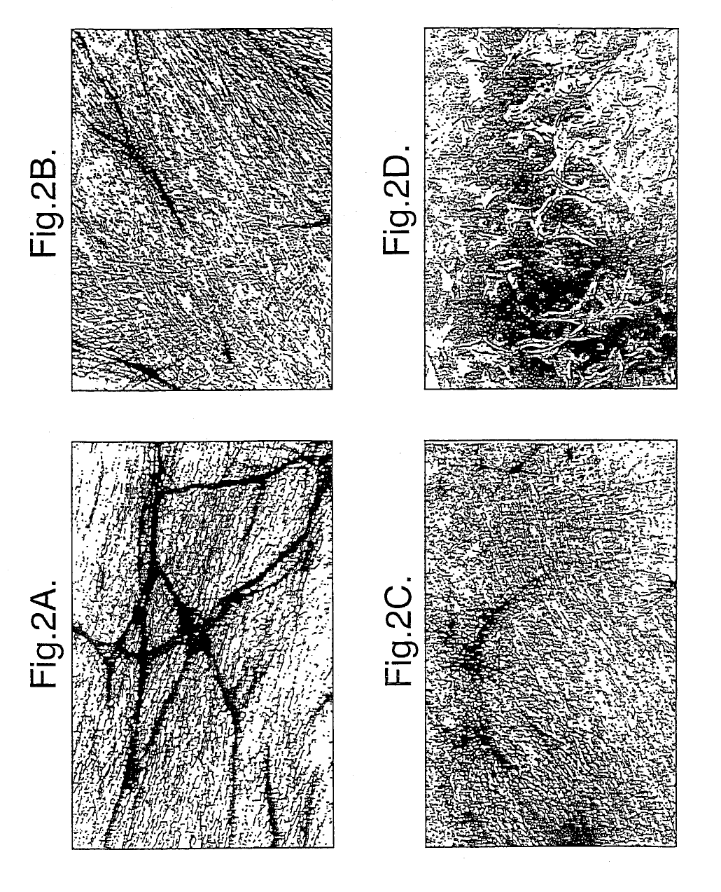

In this system HUVECs grown on a bed of fibroblast in a special medium (TCS large vessel endothelial cell medium—Cat Nos ZHM-2951 & ZHS-9845) form new blood vessels (FIG. 2, Plate A). When HUVECs are not grown in co-culture with human fibroblasts, new blood vessel formation by HUVECs does not occur. FIG. 2 plates B, C and D show the effects of increasing doses of 5×10⁻⁶ g/mil 5×10⁻⁶ g/ml and 5×10⁻⁴ g/ml of TR01 respectively on HUVEC/fibroblasts co-culture. Angiogenesis can be seen to be progressively inhibited. Similar experiments were conducted with human microvasculature endothelial cells (HMVECs) which, in this system, yield identical results to HUVECs. TR01 has little to no effect on HUVECs or fibroblasts alone. Whereas, in the absence of TR01, endothelial cell formation occurs as depicted in plate A, in the case of plate D, TR01 can be seen to cause marked destruction of growing endothelial cells.

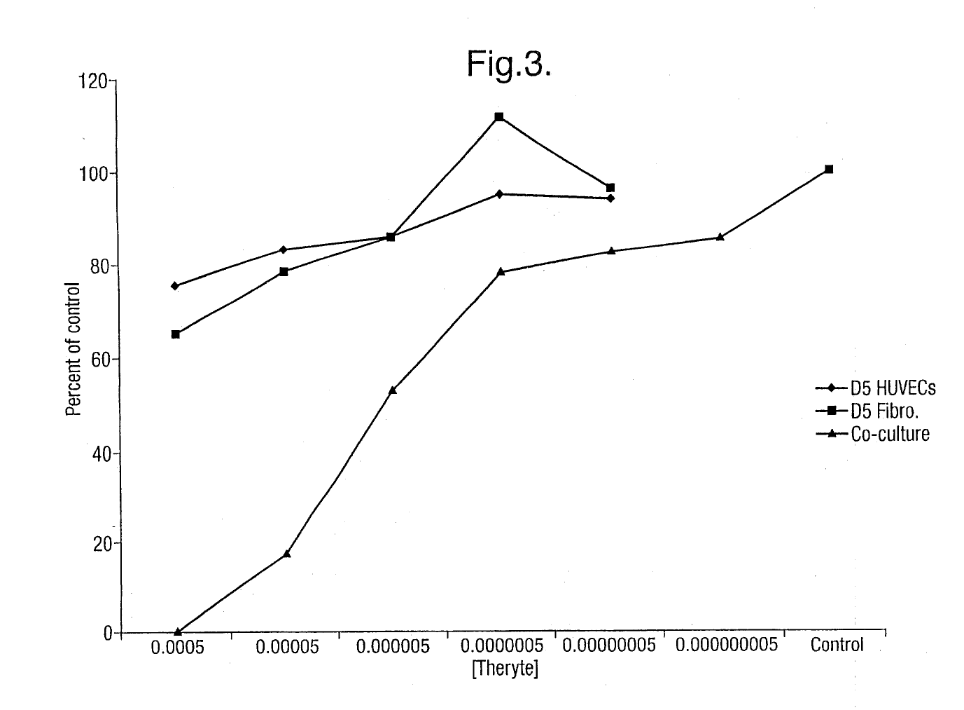

FIG. 3 shows the effect of serial tenfold dilution of TR01 (‘Theryte’—x axis) from a concentration of 0.0005 M on fibroblasts alone endothelial cells alone and co-cultures in which angiogenesis normally occurs. The toxicity for each type of culture is presented as a percentage of control cultures not exposed to TR01 (y axis). In the case of the single cultures, results are measured as cell numbers after 7 days in culture. In the angiogenesis co-cultures they are measured as a degree of tubule formation using a counting graticule system. It can be seen that TR01 is markedly selectively toxic to the angiogenic cultures as compared to the cultures of HUVECs and fibroblasts alone.

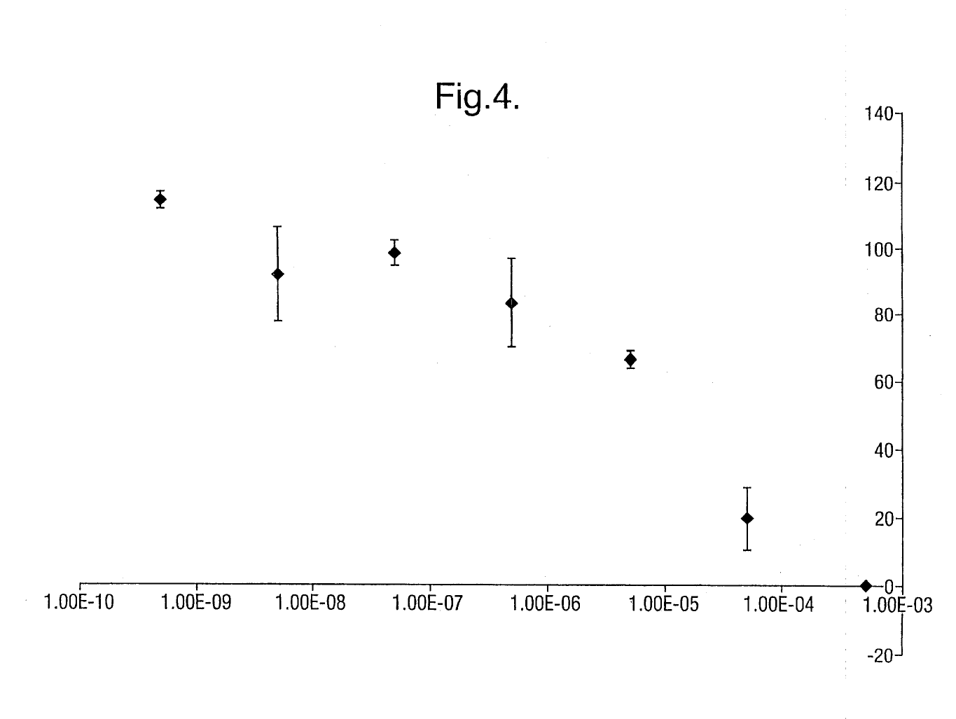

FIG. 4 confirms the toxic effect of TR01 on angiogenic cultures by three repeated, independent studies each conducted in triplicate.

### Example 2

### Effect of Polystyrene Sodium sulphonafe-co-vinyl naphthalene (TR01 TheraSol) on an in-vivo Human Colon Carcinoma Xenograft

This experiment describes the inhibitory effect of TR01 on early tumour growth in-vivo (macroscopic HT29 colonic carcinoma xenografts). The xenografts were prepared using standard techniques such as those first described by the inventor H. M. Warenius, Ph.D. thesis 1980, Cambridge University, UK.

The results are depicted in FIG. 5. These show that for growing tumours still establishing a blood supply (early rumours), TR01 has an appreciable and significant effect in slowing growth. The benefit of TR01 is similar to a five-daily intravenous injection of the maximum tolerated dose of 5-fluorouracil (the most effective drug against colon cancer at present).

Methodology

Cryopreserved HUVECs were thawed and plated into culture flasks at 2500 cells/cm². The cells were then harvested by trypsinisation, counted and seeded into 24 well plates at 2500 cells/cm² and allowed to adhere for 24 hours. The medium was then replaced with medium containing TR01 at a range of concentrations. Sufficient plates were provided to construct a growth curves for control cultures and test cultures at each concentration.

Preparation of TR01

2-vinylnaphthalene (1.62 g; 10 mmol), 4-vinylbenzylsulphonic acid butyl ester (2.4 g; 10 mmol) and 2,2′-azobis[2-methylpropionitrile] (0.67 g; 4 mmol; 20 mol. %) were placed in a 3-necked round bottomed flask, fitted with a condenser. A slow stream of nitrogen was passed through the system for fifteen minutes, and then a balloon, full with nitrogen, was fitted on top of the condenser to seal the system from air. Dry dichloromethane was added until all of the reagents had dissolved (about 60 ml). The reaction mixture was heated to 60° C. and allowed to reflux with stirring. After 21 hr of refluxing, the reaction mixture was allowed to cool to room temperature for 1 hr and was slowly added to butan-1-ol (400 ml) to produce a colourless precipitate of butyl-protected TR01, which was filtered through a sintered glass funnel (number 5) under a water pump vacuum. The residue was dried (2 days in a vacuum oven; 40° C.; 760 mmHg). The butyl ester was hydrolysed with sodium hydroxide to give TR01.

### REFERENCES

Beardsley, T. (1994) Scientific American 270, 118

Bergers, G., Javaherian, K., Lo K-M et al. (1999) Effects of angiogenesis inhibitors on multistage carcinogenesis in mice. Science 248, 808-812

Folkman, J. (1989). What is the evidence that tumours are angiogenesis dependent? J Natl. Cancer Inst. 82, 4-6.

Gasparini, G. (1997) Anti-angiogenic drugs as a novel anticancer therapeutic strategy. Which are the more promising agents? What are the clinical developments and indications? Crit. Rev. Oncol. Hematol., 26, 147-162.

Gasparini, G. (1999) The rationale and future potential of angiogenesis inhibitors in neoplasia Drugs 58, 17-38.

Jain R. K. (1998) The next frontier of molecular medicine: Delivery of Therapeutics, Nature Med., 4, 655-657.

1. A composition comprising:
(a) an active component having a therapeutic and/or diagnostic activity; and
(b) a delivery component for facilitating delivery of the active component,
wherein the delivery component has a therapeutic and/or diagnostic activity.

2. A composition according to claim 1, wherein the delivery component comprises an amphiphilic polymer, which polymer comprises hydrophilic groups and hydrophobic groups.

3. A composition according to claim 2, wherein the hydrophilic groups and/or hydrophobic groups provide the polymer with a therapeutic and/or diagnostic activity.

4. A composition according to claim 2 or claim 3, wherein the polymer comprises further groups which provide the polymer with a therapeutic and/or diagnostic activity.

5. A composition according to any of claims 2-4, wherein the polymer comprises a co-polymer formed from a monomer comprising a hydrophilic group and a co-monomer comprising a hydrophobic group.

6. A composition according to claim 5, wherein the co-polymer comprises the following structure: 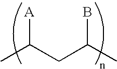
wherein A comprises a hydrophilic group, B comprises a hydrophobic group and n is an integer representing a degree of polymerisation of the polymer.

7. A composition according to any of claims 2-6, wherein the polymer comprises a co-polymer formed from a monomer comprising a hydrophilic group, a co-monomer comprising a hydrophobic group and a further co-monomer comprising a group for providing the polymer with a therapeutic and/or diagnostic activity.

8. A composition according to claim 7, wherein the co-polymer comprises the following structure: 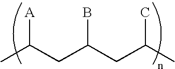
wherein A comprises a hydrophilic group, B comprises a hydrophobic group, C comprises a group for providing the polymer with a therapeutic and/or diagnostic activity and/or a group for providing the polymer with a further desirable property, and n is an integer representing a degree of polymerisation of the polymer.

9. A composition according to claim 8, wherein the group for providing the polymer with a further desirable property comprises a magnetic imaging group or a fluorescent group.

10. A composition according to any of claims 7-9, wherein the group for providing the polymer with a therapeutic and/or diagnostic activity is attached to the polymer backbone via a linkage that is hydrolisable under physiological conditions, such the group is able to be released from the polymer in vivo.

11. A composition according to any of claims 5-10, wherein the co-polymer comprises a random, block or graft co-polymer.

12. A composition according to any of claims 2-11, wherein the hydrophylic group comprises sulphonic acid derivatives such as sodium sulphonate groups, carboxylic acid derivatives such as sodium carboxylate groups, pyridyl derivatives, oligo- and poly-ether derivatives, and crown ether derivatives.

13. A composition according to any of claims 2-12, wherein the hydrophobic group comprises aromatic groups, such as phenyl derivatives and naphthyl derivatives, alkyl groups and groups comprising a heteroatom, such as heteroaromatic groups.

14. A composition according to any preceding claim, wherein the active component has a cytotoxic activity, an anti-HIV activity or an anti-angiogenic activity.

15. A composition according to any preceding claim, wherein the delivery component has a cytotoxic activity, an anti-HIV activity or an anti-angiogenic activity.

16. A composition according to any preceding claim, wherein the active component has a cytotoxic activity and the delivery component has an anti-angiogenic activity.

17. A composition according to any preceding claim, wherein the active component comprises: a taxane such as Taxol® or Taxotere®; busulphan; Hycamtin® (topotecan), Camptosar® (irinotecan); a camptothecan analogue; a toposomerase inhibitor; and/or an anthracycline analogue such as Doxorubicin® (adriamycin).

18. A composition as defined in any preceding claim for use in medicine.

19. Use of a composition as defined in any of claims 1-17 in the manufacture of a medicament effective in the prevention and/or treatment of angiogenesis.

20. Use according to claim 19, wherein the angiogenesis is associated with: vascular stents in arterioschlerosis; diabetic retinopathy; conditions following ophthalmic surgery; vascular causes of blindness; a dermatological condition; and a cosmetic condition affecting ageing of the skin.

21. Use according to claim 20, wherein: the ophthalmic surgery comprises surgery for glaucoma; the condition following ophthalmic surgery is macular degeneration; the vascular cause of blindness is macular degeneration; the dermatological condition is psoriasis; or the cosmetic condition affecting the skin is telangiectasis.

22. Use of an amphiphylic polymer for the manufacture of a medicament effective in inhibiting the activity of an angiogenic factor in the treatment of cancer.

23. Use of an amphiphylic polymer for the manufacture of a medicament effective in delivering to a treatment region: a taxane such as Taxol® or Taxotere®; busulphan; Hycamtin® (topotecan), Camptosar® (irinotecan); a camptothecan analogue; a toposomerase inhibitor; and/or an anthracycline analogue such as Doxorubicin® (adriamycin).

24. Use according to claim 22 or claim 23, wherein the amphiphilic polymer is a polymer as defined in any of claims 2-13 and 15.

25. Use according to any of claims 22-24, wherein the polymer is capable of trapping soluble angiogenic factors to prevent angiogenesis.
